(19) United States
(12) Patent Application Publication
(10) Pub. No.: US 2026/0240812 A1
(43) Pub. Date: Aug. 20, 2026

(54) TREATMENT FOR AMYOTROPHIC LATERAL SCLEROSIS (ALS)

(71) Applicant: Zydus Lifesciences Limited, Ahmedabad, Gujarat (IN)

(72) Inventors: Sameer Agarwal, Ahmedabad (IN); Deven V. Parmar, Ahmedabad, Gujarat (IN); Jain Mukul, Ahmedabad, Gujarat (IN); Abhijit Chatterjee, Ahmedabad, Gujarat (IN); Binu Philip, Ahmedabad, Gujarat (IN); Harilal Patel, Ahmedabad, Gujarat (IN); Kasinath Viswanathan, Ahmedabad, Gujarat (IN); Rajiv Sharma, Hayward, CA (US)

(21) Appl. No.: 19/489,530

(22) PCT Filed: May 23, 2024
(86) PCT No.: PCT/IB2024/055029

§ 371 Date: Dec. 2, 2025

(30) Foreign Application Priority Data
Jun. 2, 2023 (IN) 202321037929

(51) Int. Cl. A61K 31/40; A61P 21/00; A61P 25/28

(52) U.S. Cl. CPC A61K 31/40; A61P 21/00; A61P 25/28

(57) ABSTRACT

Present invention relates to the development of therapeutic compound for the treatment of Amyotrophic lateral sclerosis (ALS). Specifically, the present invention provides a NLRP3 inhibitors or its pharmaceutically acceptable salt or suitable composition useful in the treatment of Amyotrophic lateral sclerosis (ALS) and other related disorders including progressive bulbar palsy, primary lateral sclerosis, progressive muscular atrophy, spinal muscular atrophy, Kennedy's disease, and post-polio syndrome. Formula (I)

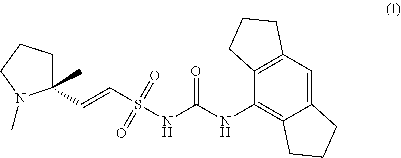 [SMILES: CN1CCC[C@]1(C)/C=C/S(=O)(=O)NC(=O)Nc1c2c(cc3c1CCC3)CCC2]

### CROSS-REFERENCE TO RELATED APPLICATIONS

This application is a U.S. national stage patent application under 35 U.S.C. § 371 of International Application No. PCT/IB2024/055029, filed on May 23, 2024, which application claims the benefit of and priority to Indian Patent Application number 202321037929, filed on Jun. 2, 2023, each of which is incorporated by reference herein in its entirety.

### FIELD OF THE INVENTION

The present invention relates to the development of therapeutic compound for the treatment of Amyotrophic lateral sclerosis (ALS). Specifically, the present invention provides a NLRP3 inhibitors or its pharmaceutically acceptable salt or suitable composition useful in the treatment of Amyotrophic lateral sclerosis (ALS) and other related disorders including progressive bulbar palsy, primary lateral sclerosis, progressive muscular atrophy, spinal muscular atrophy, Kennedy's disease, and post-polio syndrome.

### BACKGROUND OF THE INVENTION

The nucleotide-binding oligomerization domain (NOD)-like receptor family, pyrin domain-containing 3 (NLRP3 or NALP3) inflammasome are a major source of mucosal interleukin (IL)-1β and the inflammasomes are mostly subject to activation by multiple aspects of inflammation-associated stress. NLRP3 is a cytosolic pattern recognition receptor (PRR) that senses exogenous and endogenous danger signals. The NLRP3 protein is made up of three domains: a leucine-rich repeat domain (LRR), a NOD containing a caspase activation and recruitment domain (CARD) (NACHT), and a pyrin domain (PYD). Upon activation, NLRP3 oligomerizes and triggers assembly of the adapter apoptosis-associated speck-like protein containing a CARD (ASC) via PYD-PYD interactions. ASC fibrils assemble into large structures, called ASC specks, and recruit pro-caspase-1, leading to its autoproteolytic activation. The activated caspase-1 is able to cleave pro-IL-1B and pro-IL-18 to generate the inflammatory cytokines IL-1β and IL-18 (Guo et al., 2015; Dinarello et al., 2012).

Involvement of the NLRP3 inflammasome in different kinds of diseases provides new avenues to design drugs targeting NLRP3 inflammasome. To date, clinical treatment of NLRP3-related diseases targets IL-1B, with IL-1β antibodies or recombinant IL-1β receptor antagonist, such as canakinumab and anakinra, respectively. In addition, a few small-molecule compounds have shown anti-inflammatory effects on NLRP3 inflammasome activation in vitro, including MCC950, β-hydroxybutyrate (BHB), Bay 11-7082, dimethyl sulfoxide (DMSO) and type I interferon. However, most of these inhibitors are relatively nonspecific and have low efficacy. For inhibitors targeting IL-1B, it should be noted that IL-1β secretion is not the only product of NLRP3 inflammasome activation; instead, other proinflammatory cytokines, including high-mobility group box 1 (HMGB1) and IL-18 may participate in the pathogenesis of these diseases. Moreover, IL-1β can be produced by inflammasome-independent pathways or other inflammasomes. Therefore, inhibitors targeting IL-1β may lead to unintended immunosuppressive effects besides preventing NLRP3 inflammasome activation itself. Pharmacological inhibitors specific to NLRP3 inflammasome may be the best choice for treatment of NLRP3-related diseases. (Yang et al., 2019).

Amyotropic lateral sclerosis (ALS) is a devastating neurodegenerative disease that is characterised by loss of motor neurones in the spinal cord, brainstem and motor cortex [Neuropathol Appl Neurobiol 1998; 24 (2): 104-17]. ALS patients exhibit weakness of the limbs, abnormalities of speech and difficulties in swallowing. The weakness ultimately progresses to respiratory impairment and half of all patients die within 3 years of the onset of symptoms, largely due to respiratory failure. About 5-10% of all ALS patients show familial traits and 20-30% of familial ALS patients have a mutation in their copper/zinc superoxide dismutase 1 (SOD1) gene. However, in more than 90% of ALS patients the disease is sporadic and does not show any familial traits.

Amyotropic lateral sclerosis (ALS) is a rare disease across most of the world, with an incidence of 1-2 per 100,000 per year (similar to that of multiple sclerosis [Eur. J. Neurol (2006) 13:700-722] and a prevalence of 1/20,000 [JNNP (1992) 55:1116-1120; Neurology (2001) 56:239-244] These figures understate the impact of ALS, however, when one considers that the lifetime risk of developing the disease is roughly 1 in 400 [Neurol. (2006) 253:1642-1643].

There is at present no obvious consensus understanding of the pathogenic mechanism or an effective therapeutic approach for sporadic ALS, although several hypotheses, including oxidative stress, glutamate excitotoxicity, impaired axonal transport, neurofilament disintegration, mitochondrial dysfunction, neurotrophic deprivation and proteasomal dysfunction have been proposed as causal mechanisms of motor neurone degeneration [Muscle Nerve 2002; 25 (2): 135-59; Nature 2004; 427 (6977): 801]. In contrast, a subgroup of familial ALS patients, those with mutant SOD1, have initiated wide-ranging research activities.

Amyotrophic lateral sclerosis (ALS) most often starts insidiously, with painless, asymmetric weakness in distal limb muscles; a ‘bulbar onset’ in which the disease starts in speech and swallowing muscles occurs in ~30% of cases. Occasionally, there will be prominent frontotemporal dementia [J. Neurol. Sci. (2000) 180:15-20] or, more rarely, Parkinsonism. On average, patients with ALS will require a feeding tube, tracheostomy and mechanical ventilator to sustain life beyond 3 years of disease onset [Neuroepidemiology (2005) 25 (3): 114-119].

There are various approved drugs have been investigated for the treatment of amyotrophic lateral sclerosis (ALS). Based on the hypothesis that free radicals play a general role in the neurodegenerative process in motor neuron disease, selegiline has been tested in a group of patients affected by amyotrophic lateral sclerosis (ALS) to examine whether it might modify the progression of the disease. However data do not show any significant effect of selegiline in modifying the progression of ALS (Arch Neurol. 1998; 55(1): 93-96). Carbidopa/levodopa also showed mild effect to improve the spasticity in ALS patients (Clinical Trial NCT03929068).

Recently there has been a major breakthrough in the field of ALS drug discovery. FDA has approved Qalsody (tofersen) in April 2023 to treat patients with amyotrophic lateral sclerosis (ALS) associated with a mutation in the superoxide dismutase 1 (SOD1) gene (SOD1-ALS). Qalsody received priority review, orphan drug and fast track drug designations. Qalsody is an antisense oligonucleotide that targets SOD1 mRNA to reduce the synthesis of SOD1 protein. The approval was based on a reduction in plasma neurofilament light (NfL), a blood-based biomarker of axonal (nerve) injury and neurodegeneration. Thus according to a news post in Nature Reviews Drug Discovery, “NfL makes regulatory debut as neurodegenerative disease biomarker”. It says this approval established a proof of potential for this neuroscience biomarker that could have implications for other diseases of the brain as well (Nature Reviews Drug Discovery, 17 May 2023).

There are various agents in various stages of Phase 1 and Phase II development that target the NLRP3 inflammasome. (Freeman et al., 2020). Agents like MCC950, CY-09, OLT1177, Tranilast, Oridonin, NT-0167 displayed the good therapeutic properties, as they directly target NLRP3 itself, but not other components (NEK7, ASC, caspase-1, or IL-1β) up-/downstream of NLRP3 inflammasome activation. Furthermore, these inhibitors are being used in clinical practice or are being investigated at phase II clinical trials having shown relatively high safety (Yang et al., 2019).

All current therapies are limited to injectable biologics therefore, there remains an unmet clinical need for more targeted and preferably small molecule, compounds as an alternative to IL-1 targeted biologics. The present invention discloses use of compound of formula (I) which may have beneficial effects in treating patients suffering from Amyotrophic lateral sclerosis (ALS).

Various sulfonylurea based compounds as NLRP3 modulators and their process for the preparation have been disclosed in the Bioorg Med Chem Lett; 2020 Nov. 1; 30(21): 127571, which are useful in the treatment of the diseases or conditions mediated by NLRP3 or conditions in which interleukin 1β activity is implicates.

WO2023281455 discloses the use of NLRP3 modulators and its pharmaceutically acceptable salts for the treatment of Cryopyrin Associated Periodic Syndromes (CAPS).

WO2023026222 discloses the use of NLRP3 modulators and its pharmaceutically acceptable salts for the treatment of Parkinson Disease (PD).

### SUMMARY OF THE INVENTION

The present invention provides a therapeutic compound of formula (I) and their pharmaceutically acceptable salts for the prevention and treatment of Amyotrophic lateral sclerosis (ALS) and other related forms of disorders includes progressive bulbar palsy, primary lateral sclerosis, progressive muscular atrophy, spinal muscular atrophy, Kennedy's disease, and post-polio syndrome.

### EMBODIMENTS OF THE INVENTION

In an embodiment the present invention provides a therapeutic compound of formula (I)

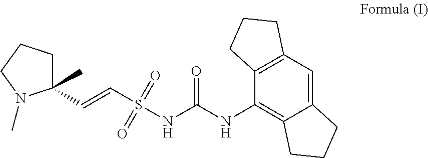 [SMILES: CN1CCC[C@]1(C)/C=C/S(=O)(=O)NC(=O)Nc1c2c(cc3c1CCC3)CCC2]

suitable for the treatment and prevention of Amyotrophic lateral sclerosis (ALS) and other related forms of disorders.

In an embodiment, the present invention provides a compound of formula (I) and its pharmaceutically acceptable salts suitable for the treatment and prevention of Amyotrophic lateral sclerosis (ALS) and other related forms of disorders.

In yet another embodiment, the present invention provides the administration of therapeutic compound of formula (I) and its pharmaceutically acceptable salts alone or in combination with suitable for the treatment and prevention of Amyotrophic lateral sclerosis (ALS) and other related forms of disorders.

In a further embodiment, the present invention provides use of compound of formula (I) or its pharmaceutically acceptable salts for the treatment and prevention of Amyotrophic lateral sclerosis (ALS) and other related forms of disorders.

In another embodiment, the present invention provides a method of treating Amyotrophic lateral sclerosis (ALS) and other related forms of disorders using pharmaceutical composition of compound of formula (I) or its pharmaceutically acceptable salts.

In yet another embodiment, the present invention provides a suitable composition comprising the compound of formula (I) or its suitable pharmaceutical compositions for the treatment and prevention of Amyotrophic lateral sclerosis (ALS) and other related forms of disorders.

The above and other embodiments of the present invention are disclosed further hereinafter.

### DETAIL DESCRIPTION OF THE INVENTION

### Definition

As used above, and throughout this disclosure, the following terms, unless otherwise indicated, shall be understood to have the following meanings:

The terms ‘treatment’ or ‘treat’ refer to slowing, stopping, or delaying the progression of the disease or clinical symptoms in a patient, as evidenced by a decrease or elimination of a clinical or diagnostic symptom of the disease, disorder or condition.

“Patient” includes both human and animals. “Mammal” means humans and other mammalian animals.

The term “preventing” refers to barring a subject from acquiring a disorder or disease in the first place.

A “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

As used herein “treating” includes achieving, partially or substantially, one or more of the following results: partially or totally reducing the extent of the disease, disorder or syndrome. Delaying, inhibiting or preventing the progression of the disease, disorder or syndrome includes for example, delaying, inhibiting or preventing the progression of Amyotrophic lateral sclerosis (ALS) and other related forms of disorders.

The present invention describes a method of treating a subject suffering from Amyotrophic lateral sclerosis (ALS) and other related forms of disorders.

In an embodiment the present invention provides a compound of formula (I) and its pharmaceutically acceptable salts suitable for the treatment or prevention of Amyotrophic lateral sclerosis (ALS) and other related forms of disorders.

In a further embodiment the present invention provides use of the compound of formula (I) or its suitable pharmaceutical compositions for the treatment or prevention Amyotrophic lateral sclerosis (ALS) and other related forms of disorders.

In an embodiment other related forms of disorders are selected from progressive bulbar palsy, primary lateral sclerosis, progressive muscular atrophy, spinal muscular atrophy, Kennedy's disease, and post-polio syndrome.

### Therapeutic Applications

The invention provides for use of compound of formula (I) and pharmaceutical compositions described herein in treating Amyotrophic lateral sclerosis (ALS). The invention also provides methods of treating Amyotrophic lateral sclerosis (ALS) by administering a compound of Formula (I) or a pharmaceutically acceptable salt thereof to a subject in need thereof.

One aspect of the present invention provides a pharmaceutical composition comprising a compound of Formula (I) or a pharmaceutically acceptable salt thereof, for use in treating Amyotrophic lateral sclerosis (ALS) wherein Formula (I) is represented by:

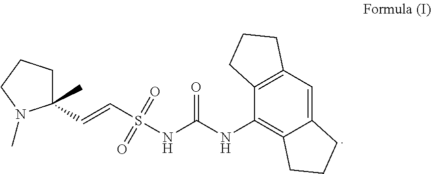 [SMILES: CN1CCC[C@]1(C)/C=C/S(=O)(=O)NC(=O)Nc1c2c(cc3c1CCC3)CCC2]

The pharmaceutical composition for use may be further characterized according to, for example, the identity of the components in the pharmaceutical composition, the route by which the pharmaceutical composition is administered to a subject, use of the pharmaceutical composition in combination with additional therapeutic agents, dosing amount of a compound of Formula (I) or pharmaceutically acceptable salt thereof, dosing frequency of the pharmaceutical composition, patients to be treated, and other features as described in more detail below.

### Therapeutic Effects

The pharmaceutical composition for use can be further characterized according to therapeutic effects. For example, in certain embodiments, the use is characterized by inhibiting progression of Amyotrophic lateral sclerosis (ALS).

The term “pharmaceutical composition” refers to a mixture of an NLRP3 antagonist or other compound described herein with other chemical components (referred to collectively herein as “excipients”) such as carriers, stabilizers, diluents, dispersing agents, suspending agents, and/or thickening agents. The pharmaceutical composition facilitates administration of the NLRP3 antagonist or other compound to an organism. Multiple techniques of administering a compound exist in the art including, but not limited to rectal, oral, and intravenous, aerosol, and parenteral, ophthalmic, pulmonary, and topical administration.

### Route of Administration

The pharmaceutical composition for use can be further characterized according to the route of administration for the pharmaceutical composition.

The compositions of the present invention can be delivered directly or in pharmaceutical compositions containing excipients, as is well known in the art. The present methods of treatment involve administration of an effective amount of a compound of the present invention to a subject having or at risk for having Amyotrophic lateral sclerosis (ALS).

An effective amount, e.g., dose, of compound or drug can readily be determined by routine experimentation, as can an effective and convenient route of administration and an appropriate formulation. Various formulations and drug delivery systems are available in the art. (See, e.g., Gennaro, ed. (2000) Remington's Pharmaceutical Sciences; and Hardman, Limbird, and Gilman, eds. (2001) The Pharmacological Basis of Therapeutics, supra.)

Suitable routes of administration may, for example, include oral, rectal, topical, nasal, pulmonary, ocular, intestinal, and parenteral administration. Primary routes for parenteral administration include intravenous, intramuscular, and subcutaneous administration. Secondary routes of administration include intraperitoneal, intra-arterial, intra-articular, intracardiac, intracisternal, intradermal, intralesional, intraocular, intrapleural, intrathecal, intrauterine, and intraventricular administration. The indication to be treated, along with the physical, chemical, and biological properties of the drug, dictate the type of formulation and the route of administration to be used, as well as whether local or systemic delivery would be preferred.

In a preferred embodiment, the present invention provides effective amount of formula (I) or its pharmaceutically acceptable salts is administered by oral route of administration.

In certain other embodiments, the pharmaceutical composition further comprises one or more pharmaceutical excipients. In certain embodiments, the pharmaceutical formulation is in the form of a tablet or capsule.

### Dosing Amount

In certain embodiments, composition for use can be further characterized according to the dose of compound administered to a subject. For example, in certain embodiments, the pharmaceutical composition for use is further characterized according to the feature that the pharmaceutical composition provides the compound of Formula (I) or a pharmaceutically acceptable salt thereof at a dose in the range of 1 mg to 500 mg with respect to compound of formula (I) for the treatment and prevention of Amyotrophic lateral sclerosis (ALS) preferably 1 mg to 250 mg and more preferably 1 mg to 150 mg with respect to compound of formula (I) for the treatment and prevention of Amyotrophic lateral sclerosis (ALS).

In certain embodiments, the compound is administered in an amount to provide compound of formula (I) or a pharmaceutically acceptable salt thereof in the range of about 1 mg to about 500 mg on each day the compound is administered to the subject.

In a preferred embodiment, the compound is administered orally in an amount to provide compound of formula (I) or a pharmaceutically acceptable salt thereof in the range of about 1 mg to about 25 mg on each day the compound is administered to the subject. In certain embodiments, the compound is administered orally in an amount to provide compound of formula (I) or a pharmaceutically acceptable salt thereof in the range of about 25 mg to about 50 mg on each day the compound is administered to the subject. In certain embodiments, the compound is administered orally in an amount to provide compound of formula (I) or a pharmaceutically acceptable salt thereof in the range of about 50 mg to about 75 mg on each day the compound is administered to the subject. In certain embodiments, the compound is administered orally in an amount to provide compound of formula (I) or a pharmaceutically acceptable salt thereof in the range of about 75 mg to about 100 mg on each day the compound is administered to the subject. In certain embodiments, the compound is administered orally in an amount to provide compound of formula (I) or a pharmaceutically acceptable salt thereof in the range of about 100 mg to about 125 mg on each day the compound is administered to the subject. In certain embodiments, the compound is administered orally in an amount to provide compound of formula (I) or a pharmaceutically acceptable salt thereof in the range of about 125 mg to about 150 mg on each day the compound is administered to the subject. In certain embodiments, the compound is administered orally in an amount to provide compound of formula (I) or a pharmaceutically acceptable salt thereof in the range of about 150 mg to about 175 mg on each day the compound is administered to the subject. In certain embodiments, the compound is administered orally in an amount to provide compound of formula (I) or a pharmaceutically acceptable salt thereof in the range of about 175 mg to about 200 mg on each day the compound is administered to the subject. In certain embodiments, the compound is administered orally in an amount to provide compound of formula (I) or a pharmaceutically acceptable salt thereof in the range of about 200 mg to about 225 mg on each day the compound is administered to the subject. In certain embodiments, the compound is administered orally in an amount to provide compound of formula (I) or a pharmaceutically acceptable salt thereof in the range of about 225 mg to about 250 mg on each day the compound is administered to the subject. In certain embodiments, the compound is administered orally in an amount to provide compound of formula (I) or a pharmaceutically acceptable salt thereof in the range of about 250 mg to about 275 mg on each day the compound is administered to the subject. In certain embodiments, the compound is administered orally in an amount to provide compound of formula (I) or a pharmaceutically acceptable salt thereof in the range of about 275 mg to about 300 mg on each day the compound is administered to the subject. In certain embodiments, the compound is administered orally in an amount to provide compound of formula (I) or a pharmaceutically acceptable salt thereof in the range of about 300 mg to about 325 mg on each day the compound is administered to the subject. In certain embodiments, the compound is administered orally in an amount to provide compound of formula (I) or a pharmaceutically acceptable salt thereof in the range of about 325 mg to about 350 mg on each day the compound is administered to the subject. In certain embodiments, the compound is administered orally in an amount to provide compound of formula (I) or a pharmaceutically acceptable salt thereof in the range of about 350 mg to about 375 mg on each day the compound is administered to the subject. In certain embodiments, the compound is administered orally in an amount to provide compound of formula (I) or a pharmaceutically acceptable salt thereof in the range of about 375 mg to about 400 mg on each day the compound is administered to the subject. In certain embodiments, the compound is administered orally in an amount to provide compound of formula (I) or a pharmaceutically acceptable salt thereof in the range of about 400 mg to about 425 mg on each day the compound is administered to the subject. In certain embodiments, the compound is administered orally in an amount to provide compound of formula (I) or a pharmaceutically acceptable salt thereof in the range of about 425 mg to about 450 mg on each day the compound is administered to the subject. In certain embodiments, the compound is administered orally in an amount to provide compound of formula (I) or a pharmaceutically acceptable salt thereof in the range of about 450 mg to about 475 mg on each day the compound is administered to the subject. In certain embodiments, the compound is administered orally in an amount to provide compound of formula (I) or a pharmaceutically acceptable salt thereof in the range of about 475 mg to about 500 mg on each day the compound is administered to the subject.

In certain other embodiments, the compound is administered orally in an amount to provide compound of formula (I) or a pharmaceutically acceptable salt thereof in the range of about 1 mg to about 25 mg on each day the compound is administered to the subject. In certain embodiments, the compound is administered orally in an amount to provide compound of formula (I) or a pharmaceutically acceptable salt thereof in the range of about 1 mg to about 50 mg on each day the compound is administered to the subject. In certain embodiments, the compound is administered orally in an amount to provide compound of formula (I) or a pharmaceutically acceptable salt thereof in the range of about 1 mg to about 75 mg on each day the compound is administered to the subject. In certain embodiments, the compound is administered orally in an amount to provide compound of formula (I) or a pharmaceutically acceptable salt thereof in the range of about 1 mg to about 100 mg on each day the compound is administered to the subject. In certain embodiments, the compound is administered orally in an amount to provide compound of formula (I) or a pharmaceutically acceptable salt thereof in the range of about 1 mg to about 125 mg on each day the compound is administered to the subject. In certain embodiments, the compound is administered orally in an amount to provide compound of formula (I) or a pharmaceutically acceptable salt thereof in the range of about 1 mg to about 150 mg on each day the compound is administered to the subject. In certain embodiments, the compound is administered orally in an amount to provide compound of formula (I) or a pharmaceutically acceptable salt thereof in the range of about 1 mg to about 175 mg on each day the compound is administered to the subject. In certain embodiments, the compound is administered orally in an amount to provide compound of formula (I) or a pharmaceutically acceptable salt thereof in the range of about 1 mg to about 200 mg on each day the compound is administered to the subject. In certain embodiments, the compound is administered orally in an amount to provide compound of formula (I) or a pharmaceutically acceptable salt thereof in the range of about 1 mg to about 225 mg on each day the compound is administered to the subject. In certain embodiments, the compound is administered orally in an amount to provide compound of formula (I) or a pharmaceutically acceptable salt thereof in the range of about 1 mg to about 250 mg on each day the compound is administered to the subject. In certain embodiments, the compound is administered orally in an amount to provide compound of formula (I) or a pharmaceutically acceptable salt thereof in the range of about 1 mg to about 275 mg on each day the compound is administered to the subject. In certain embodiments, the compound is administered orally in an amount to provide compound of formula (I) or a pharmaceutically acceptable salt thereof in the range of about 1 mg to about 300 mg on each day the compound is administered to the subject. In certain embodiments, the compound is administered orally in an amount to provide compound of formula (I) or a pharmaceutically acceptable salt thereof in the range of about 1 mg to about 325 mg on each day the compound is administered to the subject. In certain embodiments, the compound is administered orally in an amount to provide compound of formula (I) or a pharmaceutically acceptable salt thereof in the range of about 1 mg to about 350 mg on each day the compound is administered to the subject. In certain embodiments, the compound is administered orally in an amount to provide compound of formula (I) or a pharmaceutically acceptable salt thereof in the range of about 1 mg to about 375 mg on each day the compound is administered to the subject. In certain embodiments, the compound is administered orally in an amount to provide compound of formula (I) or a pharmaceutically acceptable salt thereof in the range of about 1 mg to about 400 mg on each day the compound is administered to the subject. In certain embodiments, the compound is administered orally in an amount to provide compound of formula (I) or a pharmaceutically acceptable salt thereof in the range of about 1 mg to about 425 mg on each day the compound is administered to the subject. In certain embodiments, the compound is administered orally in an amount to provide compound of formula (I) or a pharmaceutically acceptable salt thereof in the range of about 1 mg to about 450 mg on each day the compound is administered to the subject. In certain embodiments, the compound is administered orally in an amount to provide compound of formula (I) or a pharmaceutically acceptable salt thereof in the range of about 1 mg to about 475 mg on each day the compound is administered to the subject. In certain embodiments, the compound is administered orally in an amount to provide compound of formula (I) or a pharmaceutically acceptable salt thereof in the range of about 1 mg to about 500 mg on each day the compound is administered to the subject.

The amount of dose in the range of about 1 mg to about 500 mg according to present disclosure include each integer and non-integer number between a particular range.

The recitations of numerical ranges by endpoints include all numbers subsumed within that range (e.g., 1 to 5 includes 1, 1.5, 2, 2.75, 3, 3.80, 4, 5, etc.).

### Dosing Schedule

The method may be further characterized according to a dosing schedule by which the pharmaceutical composition is be administered to the subject. For example, in certain embodiments, the pharmaceutical composition is administered to the subject in the morning prior to the subject consuming food.

The compound of formula (I) or its pharmaceutically acceptable salts may be provided to the subject daily, weekly, either in a single dose or multiple dose as prescribed by physician to the person in need thereof.

In certain other embodiments, the pharmaceutical composition is administered to the subject once daily. In yet other embodiments, the pharmaceutical composition is administered to the subject once daily for at least 1 week. In yet other embodiments, the pharmaceutical composition is administered to the subject once daily for at least 2 weeks. In yet other embodiments, the pharmaceutical composition is administered to the subject once daily for at least 3 weeks. In yet other embodiments, the pharmaceutical composition is administered to the subject once daily for at least 4 weeks. In yet other embodiments, the pharmaceutical composition is administered to the subject once daily for at least 6 weeks. In yet other embodiments, the pharmaceutical composition is administered to the subject once daily for at least 8 weeks. In yet other embodiments, the pharmaceutical composition is administered to the subject once daily for at least 10 weeks. In yet other embodiments, the pharmaceutical composition is administered to the subject once daily for at least 12 weeks. In yet other embodiments, the pharmaceutical composition is administered to the subject once daily for at least 14 weeks. In yet other embodiments, the pharmaceutical composition is administered to the subject once daily for at least 16 weeks.

In another embodiment, the present invention provides a method of treating a subject suffering from Amyotrophic lateral sclerosis (ALS), which comprises treatment of a patient in need of such therapy, with compound of formula (I) or its pharmaceutically acceptable salts or suitable pharmaceutical compositions containing them.

In an embodiment, the present invention provides the combination of compound of formula (I) or its pharmaceutically acceptable salts with other suitable agents as therapeutic agent for the treatment of Amyotrophic lateral sclerosis (ALS).

In an embodiment, the additional therapeutic agent used is selected from Inhibitors of interleukin-1β (e.g. Rilonacept, Canakinumab, and Anakinra); immune-suppressants (e.g., Methotrexate, Mercaptopurine, Cyclophosphamide), metabolic disorders drugs, glucocorticoids, non-steroidal anti-inflammatory drugs, Gasdermin D inhibitors (e.g., Necrosulfonamide); Cox-2 specific inhibitors, TNF-α binding proteins (e.g., Infliximab, Etanercept), Interferon-13, Interferon, Interleukin-2, antihistamines, beta-agonist, BTK inhibitors, anticolinergics, anti-cancer agents; anti-viral drugs, for example: Remdesivir, Lopinavir/Ritonavir, Favipiravir, Molnupiravir, Tamiflu; anti-malarial agents, for example: Choloroquinone, Hydroxyl Chloroquinone; or their suitable pharmaceutically acceptable salts. Further examples for use in combination with Non-Alcoholic Steato-Hepatitis (NASH) and fibrosis drugs; anticancer; antibiotics, for example Azithromycin; hormones, Aromatase inhibitors, Colchicine, Anticoagulants, antibodies, cytokines, anti-IL6 drugs; Antiparasitics; vaccines; Interferons; drug conjugates; Drugs originally developed for SARS (ACE2 protein decoy); Intravenous vitamin C; inhibitors of mitogen-activated protein kinase signaling (ex: BAY 43-9006); Syk inhibitors; mTOR inhibitors; antibodies (Rituxan); and BCR/ABL antagonist.

The compound of formula (I) of the present invention or its pharmaceutically acceptable salts may be used further in combination with one or more suitable pharmaceutically active agents selected from following therapeutic agents in any combinations. MAO B inhibitors, selegiline (Zelapar), rasagiline (Azilect) and safinamide (Xadago); Catechol O-methyltransferase (COMT) inhibitors, Entacapone (Comtan) and opicapone (Ongentys); benztropine (Cogentin), trihexyphenidyl, Amantadine; cholinesterase inhibitors, donepezil (Aricept), galantamine (Razadyne) and rivastigmine (Exelon), Memantine (Namenda), aducanumab (Aduhelm); Riluzole (Rilutek), Edaravone (Radicava); ocrelizumab (Ocrevus), prednisone and methylprednisolone; tetrabenazine (Xenazine) and deutetrabenazine (Austedo), haloperidol (Haldol) and fluphenazine, risperidone (Risperdal), olanzapine (Zyprexa) and quetiapine (Seroquel), levetiracetam (Keppra, Elepsia XR, Spritam) and clonazepam (Klonopin); citalopram (Celexa), escitalopram (Lexapro), fluoxetine (Prozac, Sarafem) and sertraline (Zoloft), quetiapine (Seroquel), risperidone (Risperdal) and olanzapine (Zyprexa), divalproex (Depakote), carbamazepine (Carbatrol, Epitol) and lamotrigine (Lamictal).

The compounds and compositions of the present invention are also intended for use with general care provided patients with Arenaviridae viral infections, including parenteral fluids (including dextrose saline and Ringer's lactate) and nutrition, antibiotic (including Metronidazole and Cephalosporin antibiotics, such as Ceftriaxone and Cefuroxime) and/or antifungal prophylaxis, fever and pain medication, antiemetic (such as Metoclopramide) and/or antidiarrheal agents, vitamin and mineral supplements (including Vitamin C or/and K and zinc sulfate), anti-inflammatory agents (such as Ibuprofen), pain medications, and medications for other common diseases in the patient population, such anti-malarial agents (including Artemether and Artesunate-lumefantrine combination therapy), typhoid (including quinolone antibiotics, such as Ciprofloxacin, macrolide antibiotics, such as Azithromycin, cephalosporin antibiotics, such as Ceftriaxone, or aminopenicillins, such as Ampicillin), or shigellosis.

In yet another embodiment, compound of formula (I) or its pharmaceutically acceptable salts is provided in the form of pharmaceutical composition.

In an embodiment, present invention provides a pharmaceutical composition comprising compound of formula (I) or its pharmaceutically acceptable salts for treatment of Amyotrophic lateral sclerosis (ALS), wherein compound of formula (I) is

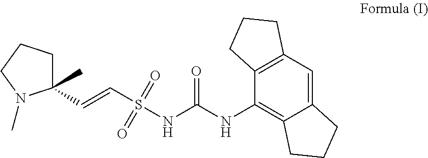 [SMILES: CN1CCC[C@]1(C)/C=C/S(=O)(=O)NC(=O)Nc1c2c(cc3c1CCC3)CCC2]

In an embodiment, the present invention provides pharmaceutical composition comprising compound of formula (I) or its pharmaceutically acceptable salts and suitable pharmaceutically acceptable excipients for the treatment of Amyotrophic lateral sclerosis (ALS).

In an embodiment, the pharmaceutically acceptable excipients described in the present invention are selected at least one from diluents, carriers, binders, disintegrating agents, lubricating agents, encapsulating agent and the like.

Diluents include, but are not limited to microcrystalline cellulose, polymethacrylates selected from Eudragit, potassium chloride, sulfobutylether b-cyclodextrin, sodium chloride and spray dried lactose, combinations thereof and other such materials known to those of ordinary skill in the art.

Carriers include, but are not limited to lactose, white sugar, sodium chloride, glucose, urea, starch, calcium carbonate and kaolin, crystalline cellulose and silicic acid, combinations thereof and other such materials known to those of ordinary skill in the art.

Binders include, but are not limited to carbomers selected from carbopol, gellan, gum Arabic, hydrogenated vegetable oil, polymethacrylates selected from Eudragit, xanthan, lactose and Zein, pregelatinized starch, combinations thereof and other such materials known to those of ordinary skill in the art.

Disintegrating agents include, but are not limited to, pregelatinized starch, bicarbonate salt, chitin, gellan gum, polacrillin potassium and docusate sodium, combinations thereof and other such materials known to those of ordinary skill in the art.

Lubricating agents used include, but are not limited to, magnesium stearate, glycerin behenate, hydrogenated vegetable oil, sodium stearyl fumarate and myristic acid, combinations thereof and other such materials known to those of ordinary skill in the art.

Encapsulating agent used include, but are not limited to, empty hard gelatin capsule shells.

The present invention is further exemplified by the following non-limiting examples.

### Composition of Compound of Formula (I)

TABLE 1

| | |
|---|---|
| Sr. | Qty/capsule (mg) |

| No. | Formulations | 25 mg | 50 mg |
|---|---|---|---|
| 1 | Compound (I) | 25 | 50.00 |
| 2 | Microcrystalline cellulose | 126 | 252.00 |
| | (Comprecel M101D+) | | |
| 3 | Pregelatinized starch | 12.3 | 24.60 |
| | (Starch 1500) | | |
| Binder solution | | | |
| 4 | Purified water | Q.S. | Q.S. |
| Lubrication | | | |
| 5 | Mg. stearate | 1.7 | 3.40 |
| | Total | 165 | 330.00 |
| 6 | Empty hard gelatin | Size ‘3’ | Size ‘0’ |
| | capsule shells | | |

TABLE 2
provides Stability data of composition as mentioned in Table 1

| | | | | | | | | | | | | | |
|---|---|---|---|---|---|---|---|---|---|---|---|---|---|
| Initial | 0.02 | 0.04 | ND | 0.06 | 0.21 | 0.11 | 0.04 | 0.03 | 0.04 | 0.30 | ND | 0.04 | Total |
| | | | | | | | | | | | | | impurities: |
| | | | | | | | | | | | | | 0.89 |
| 30 days | 0.04 | 0.05 | 0.02 | 0.07 | 0.19 | 0.09 | 0.02 | 0.02 | 0.05 | ND | 0.64 | 0.08 | Total |
| 40° C./ | | | | | | | | | | | | | impurities: |
| 75% RH | | | | | | | | | | | | | 1.27 |

The stable pharmaceutical composition may be made by dry mixing, wet granulation or dry granulation methods by techniques known to persons skilled in the art. Thus, for example,

In wet granulation process, the drug is mixed with one or more pharmaceutical excipients and granulated with suitable binding solution as described earlier, to form wet granules, the wet granules are dried and optionally sieved. The dried granules are mixed with one or more suitable excipients from those described elsewhere and then compressed into tablets or filled into capsules.

In dry mixing process, the drug is mixed with all the pharmaceutical excipients required. The blend is mixed with one or more suitable excipients from those described elsewhere and then final blend is either compressed into tablets or filled in capsules.

In dry granulation process, the drug is mixed with one or more pharmaceutical excipients and compressed into slugs and these slugs are passed through required sieve. The sieved granules are mixed with one or more suitable excipients from those described elsewhere and then compressed into tablets or filled into capsules.

One or more solvents or vehicle used in the formulation are selected from water, acetone, chloroform, dichloromethane, ethyl alcohol, ethyl acetate, methyl alcohol, isopropyl alcohol and combinations thereof and other such materials known to those of ordinary skill in the art.

The present invention further discloses use of said compound of formula (I) or their suitable pharmaceutical compositions for the treatment of Amyotrophic lateral sclerosis (ALS) and other related disorders.

In another embodiment, the present invention provides a method of treating Amyotrophic lateral sclerosis (ALS) using pharmaceutical composition of compound of formula (I) or its pharmaceutically acceptable salts. In a preferred embodiment, a method of treating Amyotrophic lateral sclerosis (ALS) using compound of formula (I) or its pharmaceutical composition.

In another embodiment of the present invention provides a process for the preparation of a stable pharmaceutical composition of compounds of formula (1) or its pharmaceutically acceptable salts.

The compound of formula (I) was prepared as per the processes disclosed in the prior art such as those mentioned elsewhere in the specification.

The invention now being generally described, will be more readily understood by reference to the following examples, which are included merely for purposes of illustration of certain aspects and embodiments of the present invention, and are not intended to limit the invention.

### Example—1

Experimental autoimmune Encephalitis: The effect of formula (I) (ZYIL1) is investigated in EAE model in mice. Female C57BL/6 (15 to 17-weeks old) mice received subcutaneously with 200 μl of emulsion (at two sites, 100 μl/site) of MOG (Sigma Aldrich) solution (2 mg/ml in PBS) in complete Freund's adjuvant (Difco Laboratories, Detroit, MI) in 1:1 ratio. Mice received intraperitoneal injections of 200 ng pertussis toxin after 2 and 48 hours post immunization. Each group comprised of seven animals. Four groups of animals are treated twice daily for 20 days with various doses (3, 10, 30 and 60 mg/kg, p.o.) of formula (I) (ZYIL1). Vehicle control group animals received vehicle only. Animals are examined daily, and clinical scores are assigned on a scale of 0-5 as per criteria given in following table. Body weight of animals is recorded once weekly. On day 22, blood samples of the animals are collected under isofluorane anesthesia. Then the animals are sacrificed, brain tissues are collected and snap frozen. Neurofilament light chain (NfL) concentration is measured in the serum and brain homogenate.

### Example—2

β-N-methylamino-L-alanine (L-BMAA) induced ALS: Male SD rats of 21 days old are subjected to 300 mg/kg dose of β-N-methylamino-L-alanine (L-BMAA) daily via intraperitoneal route for 14 days. Different groups of animals are treated with various doses of formula (I) or vehicle twice daily. Blood samples of each animals are collected at an interval of 14 days till 90 days from the day of initiation. At the end of the study, brain tissues are collected and snap frozen. Neurofilament light chain (NfL) concentration is measured in the serum and brain homogenate.

### Example—3

Efficacy, Safety, Tolerability, Pharmacokinetics and Pharmacodynamics of Formula (I) in Patients with Amyotrophic Lateral Sclerosis
Protocol Title: A phase 2, proof-of-concept, placebo controlled, randomized, double blind study of compound of formula (I) to evaluate the efficacy, safety, tolerability, pharmacokinetics and pharmacodynamics in patients with Amyotrophic Lateral Sclerosis (ALS).
Study design: A phase 2, proof-of-concept, placebo controlled, randomized, double blind, 12-week study of compound of formula (I) to evaluate the efficacy, safety, pharmacokinetics, and pharmacodynamics in patients with Amyotrophic Lateral Sclerosis (ALS).
Study population: Male or female patients aged between 18-80 years (both inclusive at screening) with diagnosis of Amyotrophic Lateral Sclerosis who meet eligibility criteria
Number of Patients: Approximately 24 patients (06 patients in each arm) are enrolled.
Duration of Patient Participation: 114±5 Days (Including 28 days of screening)

### Primary Objective:

To measure the efficacy of the treatment using the slope of progression with the revised Amyotrophic Lateral Sclerosis Functional Rating Scale (ALSFRS-R)

### Secondary Objectives:

To confirm the safety and tolerability of compound of formula (I) in patients with ALS To evaluate pharmacokinetics of compound of formula (I)
To evaluate effect of compound of formula (I) on neurofilament biomarkers

### Primary Outcome Measures:

Change from baseline in the ALSFRS-R total score [Time Frame: From baseline to Week 12]

### Secondary Outcome Measures:

Time from baseline to the occurrence of either death, or permanent assisted ventilation (>22 hours daily for >7 consecutive days), whichever comes first [Time Frame: Up to Week 12]
Change from baseline in slow vital capacity (SVC)—[Time Frame: From baseline to Week 12]
Change from baseline in serum neurofilament light chain, biomarkers—[Time Frame: From baseline to Week 12]
Number of patients with treatment emergent adverse events (TEAE) and Serious adverse event (SAE)—[Time Frame: Up to Week 12]
Assessment of pharmacokinetic parameter plasma concentration of compound of formula (I) [Time Frame: From baseline to Week 12]

### Study Period:

This is a proof-of-concept, placebo controlled, randomized, double blind study designed to evaluate the efficacy, safety, tolerability, pharmacokinetics, and pharmacodynamics in patients with Amyotrophic Lateral Sclerosis following a twice daily oral administration of compound of formula (I) or matching placebo to patients aged between 18 and 80 years (inclusive at screening).

Twenty-four (24) patients are randomly assigned in a 1:1:1:1 ratio to oral twice daily capsules of formula (I) 25 mg or formula (I) 50 mg or formula (I) 75 mg or matching placebo.

Treatment duration are twelve (12) weeks. Clinic visits is occur at Screening, Baseline, Week 4 (day 28), Week 8 (day 56), Week 12 (day 84).

The study is conducted with four treatment arms (06 participants per each arm):
Arm 1: formula (I) capsule 25 mg for oral administration
Arm 2: formula (I) capsule 50 mg for oral administration
Arm 3: formula (I) capsule 75 mg for oral administration
Arm 4: Placebo capsule for oral administration

All visit windows are consecutive calendar days and are calculated from the day the subject starts study treatment (Day 1, the day of the Baseline Visit). Any change from this visit window is considered an out of window visit deviation.

Safety data reviewed is adverse event (AE) incidence and severity, clinical laboratory results (hematology, biochemistry), electrocardiograms (ECGs), physical examinations, and vital signs.

### Test Product:

Formula (I) capsule 25 mg for oral administration
Formula (I) capsule 50 mg for oral administration
Formula (I) capsule 75 mg for oral administration

### Placebo:

Capsules for oral administration
Capsules are matched to formula (I) with respect to size, shape, and appearance

### Criteria for Evaluation:

The following safety parameters are evaluated during the study.
1. Physical examination
2. Neurological examination
3. Vital signs
4. Clinical laboratory tests
5. 12-lead ECG
6. AEs/SAEs

TABLE 3

| | | | | | | |
|---|---|---|---|---|---|---|
| IN0040001 | 34 | 34 | 29 | NA | NA | NA |
| IN0040002 | 27 | 27 | 20 | 19 | 19 | −8 |
| IN0040004 | 33 | 33 | 33 | 33 | 33 | 0 |
| IN0040003 | 26 | 26 | 26 | 24 | 25 | −1 |
| IN0020001 | 41 | 41 | 42 | 42 | 42 | 1 |
| IN0040005 | 37 | 31 | 31 | 31 | 32 | 1 |
| IN0020003 | 44 | 44 | 42 | 41 | 41 | −3 |
| IN0010001 | 36 | 36 | 36 | 36 | 28 | −8 |
| IN0100001 | 43 | 43 | 43 | 42 | 42 | −1 |
| IN0050001 | 39 | 41 | 45 | 46 | 45 | 4 |

### Result

Based on the data as shown in table 3 above, it is observed that in few patients disease progression by −8 points at the end of 12 weeks. It is also observed that improvement in ALSFRS-R score by 1 to 4 in few patients and also stable score in ALSFRS-R over 12 weeks.

### CONCLUSION

In the study it is observed that stable and improved ALSFRS-R score in some patients shows possibility of formula (I) as a therapeutic option in ALS.

While the present invention has been described in terms of its specific embodiments, certain modifications and equivalents will be apparent to those skilled in the art and are intended to be included within the scope of the present invention.

1. A method of treating Amyotrophic lateral sclerosis (ALS) or a related disorder in a subject in need thereof, wherein the method comprises administering to the subject a therapeutically effective amount of a compound of Formula (I): 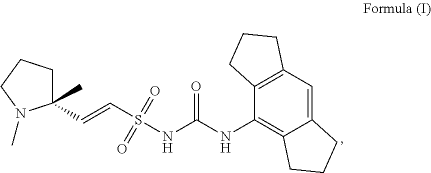 [SMILES: CN1CCC[C@]1(C)/C=C/S(=O)(=O)NC(=O)Nc1c2c(cc3c1CCC3)CCC2]
or a pharmaceutically acceptable salt thereof.

2. The method of claim 1, wherein the related disorder is selected from progressive bulbar palsy, primary lateral sclerosis, progressive muscular atrophy, spinal muscular atrophy, Kennedy's disease, and post-polio syndrome.

3. The method of claim 1, wherein the therapeutically effective amount of the compound of Formula (I) or its a pharmaceutically acceptable salt thereof is 1 mg to 500 mg.

4. The method of claim 1, wherein the compound of Formula (I) or a pharmaceutically acceptable salt thereof is administered by oral, topical or parenteral route of administration.

5. The method of claim 1, wherein the compound of Formula (I) or a pharmaceutically acceptable salt thereof is administered in combination with an additional therapeutic agent.

6. The method of claim 1, wherein the compound of Formula (I) or a pharmaceutically acceptable salt thereof is administered in the form of a pharmaceutical composition.

7-12. (canceled)

13. The method of claim 6, wherein the pharmaceutical composition comprises a pharmaceutically acceptable excipient.

14-15. (canceled)

16. The method of claim 13, wherein the pharmaceutically acceptable excipient is selected from a diluent, a carrier, a binder, a disintegrating agent, a lubricating agent, and an encapsulating agent.

17. The method of claim 16, wherein the diluent is microcrystalline cellulose or polymethacrylate.

18. The method of claim 16, wherein the carrier is selected from lactose, white sugar, sodium chloride, glucose, urea, starch, calcium carbonate and kaolin, crystalline cellulose, and silicic acid.

19. The method of claim 16, wherein the binder is selected from carbopol, gellan, gum Arabic, hydrogenated vegetable oil, and polymethacrylate.

20. The method of claim 16, wherein the disintegrating agent is selected from pregelatinized starch, bicarbonate salt, chitin, gellan gum, polacrillin potassium, and docusate sodium.

21. The method of claim 16, wherein the lubricating agent is selected from magnesium stearate, glycerin behenate, hydrogenated vegetable oil, sodium stearyl fumarate, and myristic acid.

22. The method of claim 16, wherein the encapsulating agent is empty hard gelatin capsule shell.

23. (canceled)

24. The method of claim 1, wherein the therapeutically effective amount of the compound of Formula (I) or a pharmaceutically acceptable salt thereof is 1 mg to 250 mg.

25. The method of claim 1, wherein the therapeutically effective amount of the compound of Formula (I) or a pharmaceutically acceptable salt thereof is 1 mg to 150 mg.

26. The method of claim 1, wherein the compound of Formula (I) or a pharmaceutically acceptable salt thereof is administered orally.

27. The method of claim 13, wherein the pharmaceutically acceptable excipient is selected from Eudragit, potassium chloride, sulfobutylether b-cyclodextrin, sodium chloride and spray dried lactose.

28. A method of treating Amyotrophic lateral sclerosis (ALS) in a subject in need thereof, wherein the method comprises administering to the subject a therapeutically effective amount of a compound of Formula (I): 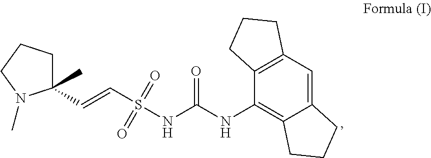 [SMILES: CN1CCC[C@]1(C)/C=C/S(=O)(=O)NC(=O)Nc1c2c(cc3c1CCC3)CCC2]
or a pharmaceutically acceptable salt thereof.

29. The method of claim 28, wherein the compound of Formula (I) or a pharmaceutically acceptable salt thereof is administered orally.
